SEQUENCE LISTING (2 sequences)

SEQ ID NO: 1 - DNA, 25 bp, Artificial Sequence
  PNprobe
tcaatctcgggaatctcaatgttag

SEQ ID NO: 2 - DNA, 25 bp, Artificial Sequence
  PNtarget
ctaacattgagattcccgagattga

(19) United States
(12) Patent Application Publication
(10) Pub. No.: US 2004/0209252 A1
(43) Pub. Date: Oct. 21, 2004

(54) Electroactive complex, electroactive probe and preparation method

(76) Inventor: Francis Garnier, Champigny Sur Marne (FR)

Correspondence Address: Oliff & Berridge, PO Box 19928, Alexandria, VA, 22320, US

(21) Appl. No.: 10/257,783

(22) PCT Filed: Apr. 23, 2001
(86) PCT No.: PCT/FR01/01241

(51) Int. Cl. C12Q 1/68; G01N 33/53; C08G 63/48; C08G 63/91; G01N 27/26

(52) U.S. Cl. 435/6; 435/7.1; 205/777.5; 525/54.1; 525/54.2

(57) ABSTRACT

The invention concerns an electroactive complex, consisting of an electroactive homopolymer or copolymer polymer of at least two monomers, and anti-ligand and a ligand having specifically interacted with said antiligand, and further at least an electron donor group, and an electroactive probe, consisting of said polymer said antiligand capable of interacting specifically with said ligand and at least an electron donor group.

The invention relates to organic electrodes produced from electroactive polymers on which antiligands intended to interact specifically with ligands are bonded.

The specific interaction of the antiligand with the ligand involves a substantial and selective variation in the electrochemical properties of the electroactive polymer, such as a decrease in the electroactivity of said polymer. This variation, which depends on the concentration of grafted ligand, is observed, possibly measured, and directly correlated with the amount of ligand grafted. One of the essential applications of this technique therefore resides in the detection, identification and possibly assay of a ligand present in a biological specimen.

The aforementioned variation is of the potentiometric type, such as a variation in the oxidation potential of the electroactive polymer before and after interaction, or of the amperometric type, such as a variation in the oxidation or reduction current of the polymer before and after hybridization, determined at a defined potential.

To specifically characterize the electrochemical response of the polymer, this must have a high electroactivity.

It is therefore one of the aims of the invention to provide a probe based on an electroactive polymer to which at least one antiligand is attached, this probe possessing a high electroactivity.

Document WO-A-95/29199 teaches a polypyrrole formed from monomers each consisting of a pyrrole ring covalently substituted on the carbon in the 3 position of the pyrrole ring with a probe polynucleotide.

The polypyrrole thus obtained is applied for the detection and possibly assay of ligands in vitro or in vivo.

However, there is always the expectation of producing polymers possessing better electroactive properties.

The invention provides a modified electroactive polymer carrying at least one antiligand, the sensitivity of which electroactive polymer may be of the order of one hundred times higher than that of a known modified electroactive polymer, such as a polypyrrole forming the subject matter of document WO-A-95/29199.

Thus, a first subject of the invention is an electroactive complex consisting of an electroactive, homopolymer or copolymer, polymer of at least two monomers, an antiligand and a ligand that has specifically interacted with said antiligand, said complex furthermore including at least one electron-donating group.

According to the invention, the electron-donating group is advantageously chosen from ferrocene, quinone and derivatives of these and/or the electroactive polymer is preferably chosen from polypyrrole, polyacetylene, polyazine, poly(p-phenylene), poly(p-phenylene vinylene), polypyrene, polythiophene, polyfuran, polyselenophene, polypyridazine, polycarbazole, polyaniline and double-stranded polynucleotides.

Before explaining the invention in detail, certain terms employed in the description and the claims are defined below.

The term “electron-donating group” is understood to mean a redox pair, having a narrow, rapid and reversible, oxidation wave, such as ferrocene, quinone and their derivatives, for example their substituted derivatives.

The terms “antiligand” and “ligand” refer not only to biological molecules such as polynucleotides or peptides, but also to chemical molecules.

The antiligand is capable of interacting specifically with the ligand to form a ligand/antiligand conjugate. As examples of conjugates, mention may be made of any peptide/antibody pair, antibody/haptene pair, hormone/receptor pair, polynucleotide/polynucleotide pair, polynucleotide/nucleic acid pair and the like.

The term “polynucleotide” as employed according to the invention denotes a linked sequence of at least five nucleotides (deoxyribonucleotides or ribonucleotides), whether natural or modified, capable of being hybridized, under appropriate hybridization conditions, with an at least partially complementary polynucleotide. The term “modified nucleotide” is understood to mean, for example, a nucleotide having a modified base and/or having a modification at the internucleotide bond and/or in the backbone. As examples of modified bases, mention may be made of inosine, 5-methyldeoxycytidine, 5-dimethylaminodeoxyuridine, 2,6-diaminopurine and 5-bromodeoxyuridine. To illustrate a modified internucleotide bond, mention may be made of phosphorothioate, H-phosphonate and alkyl phosphonate bonds. Alpha-oligonucleotides, such as those described in FR-A-2 607 507 and the PNAs forming the subject of the article by M. Egholm et al., J. Am. Chem. Soc. (1992), 114, 1895-1897, are examples of polynucleotides consisting of nucleotides whose backbone is modified.

The term “peptide” means in particular any linked sequence of at least two amino acids, such as protein, protein fragment or oligopeptide, which has been extracted, separated, isolated or synthesized, such as a peptide, obtained by chemical synthesis or by expression in a recombinant organism. Also included is any peptide in the sequence of which one or more amino acids of the L series are replaced with one or more amino acids of the D series, and vice versa; any peptide at least one of whose CO—NH bonds is replaced with an NH—CO bond; any peptide at least one of whose CO—NH bonds is replaced with an NH—CO bond, the chirality of each aminoacyl residue, whether or not involved in one or more of said CO—NH bonds, being either preserved, or reversed with respect to the aminoacyl residues constituting a reference peptide (or immunoretroids); and any mimotope.

To illustrate the various classes of peptides in question, mention may be made of adrenocorticotropic hormones or fragments thereof, angiotensin analogs and inhibitors thereof, natriuretic peptides, bradykinin and peptide derivatives thereof, chemiotactic peptides, dynorphin and derivatives thereof, endorphins and derivatives thereof, enkephalins and derivatives thereof, enzyme inhibitors, fibronectin fragments and derivatives thereof, gastro-intestinal peptides, opioid peptides, oxytocin, vasopressin, vasotocin and derivatives thereof, and kinase proteins.

The term “antibody” defines any monoclonal or polyclonal antibody, any fragment of a said antibody such as the Fab, Fab′2 or Fc fragments, and any antibody obtained by genetic modification or recombination.

The terms “graft”, “bond” and “attach” in the absence of any indication are employed in the present text to denote any relationship between two entities, without defining the chemical nature thereof. It may thus be a weak bond or a covalent bond.

A linking group according to the invention links, by a covalent bond, two chemical entities after interaction of said two entities, at least one having been preactivated or activatable, for the purpose of this iinteraction, by an activated or activatable group. The linking group may therefore result from the reaction of a said activated or activatable group of one entity on a reactive functional group of the other entity, and vice versa, or from the reaction of a said activated or activatable group of one entity on another said activated or activatable group of the other entity.

The term “activated group” is understood to mean a group allowing, by its agency, the interaction of the entity to which it is attached with another entity. As an example, this may be an activated ester group such as the —CO—[O—N-phthalimide] group. The term “activatable group” is understood to mean a group which can be converted into an activated group, for example under certain reaction conditions or when brought into contact with an activated group capable of interacting with it.

The complex of the invention as defined above advantageously satisfies the following characteristics considered individually or in combination.

The ligand and the antiligand are biological molecules, especially chosen from polynucleotides and polypeptides, which may be labeled by a tracer capable of generating a signal directly or indirectly.

The complex has one of the following structures: the electron-donating group is linked, directly or indirectly, on one side to the electroactive polymer and on the other side to the antiligand or ligand, or else the electron-donating group is linked, directly or indirectly, to the antiligand, said antiligand itself being linked, directly or indirectly, to the electroactive polymer, or else the electron-donating group is linked, directly or indirectly, to the ligand that has interacted with the antiligand, said antiligand being linked, directly or indirectly, to the electroactive polymer.

When the electron-donating group is linked to the electroactive polymer, it is preferably linked covalently via a first linking group.

When the antiligand or ligand is linked to the electron-donating group and/or to the electroactive polymer, they are avantageously linked covalently via a second and a third linking group, respectively.

The first and/or second and/or third linking groups link, respectively, the electron-donating group to the electroactive polymer, the electron-donating group to the antiligand or to the ligand, and the antiligand or the ligand to the electroactive polymer, via a coupling arm.

In the structure of the complex of the invention, in which the electron-donating group is linked to the ligand, it may also be linked via an inert support, for example a polystyrene bead, a magnetic bead or a glass bead, or via a biological support, such as a cell in which the electron-donating group has been internalized.

According to a preferred complex of the invention, the electroactive polymer is a polypyrrole formed from at least two monomers each consisting of a pyrrole ring, and the electron-donating group is ferrocene, and in particular the antiligand is a probe polynucleotide and the ligand is a target polynucleotide, at least partly hybridized with said antiligand.

This preferred complex furthermore has the following characteristics, considered individually or in combination.

The ferrocene is linked, on one side, to the probe polynucleotide and on the other side to the pyrrole ring of a polypyrrole monomer, in which case the probe polynucleotide is attached to the carbon in the 1 position of one of the cyclopentadiene rings of the ferrocene, and the ferrocene is attached to the pyrrole ring via the carbon in the 1′ position of the other ring of the cyclopentadiene, or else the probe polynucleotide is linked to the ferrocene and to the pyrrole ring of said monomer at least, in which case the probe polynucleotide is attached to the carbon in the 1 position of one of the cyclopentadiene rings of the ferrocene.

The pyrrole ring is preferably substituted on the carbon in the 3 position.

The polypyrrole is a copolymer and comprises a monomer whose pyrrole ring is substituted with a —CH₂—COOH or —CH₂—CH₂OH group.

Avantageously, the first linking group between the ferrocene and the pyrrole ring is the —CONH—CH₂— group and/or the second linking group between the ferrocene and the probe polynucleotide or target polynucleotide is the —CO— group and/or the third linking group between the probe polynucleotide or target polynucleotide and the pyrrole ring is the —CH₂—CO— group.

The first and/or second and/or third linking groups which link, indirectly, the ferrocene to the pyrrole ring, the ferrocene to the probe polynucleotide or target polynucleotide, and the pyrrole ring to the probe polynucleotide or target polynucleotide, respectively, may do so via a coupling arm. The latter is advantageously a saturated hydrocarbon chain having at least two carbon atoms, preferably at least 2 or 3 carbon atoms.

The probe polynucleotide or the target polynucleotide is attached to the ferrocene and/or to the pyrrole ring of the monomer at least, via, respectively, the second and/or the third linking groups and via at least one of the amino functional groups of the probe polynucleotide or target polynucleotide.

Another object of the invention is an electroactive probe consisting of an electroactive, homopolymer or copolymer, polymer of at least two monomers, an antiligand capable of interacting specifically with a ligand, said probe furthermore including at least one electron-donating group.

Advantageously, said probe possesses at least one of the following features.

The electroactive polymer is chosen from polypyrrole, polyacetylene, polyazine, poly(p-phenylene), poly(p-phenylene vinylene), polypyrene, polythiophene, polyfuran, polyselenophene, polypyridazine, polycarbazole, polyaniline and double-stranded polynucleotides and/or the electron-donating group is chosen from ferrocene, quinone and derivatives of these, and/or the antiligand is a biological molecule, especially chosen from polynucleotides and polypeptides, and is optionally labeled with a tracer capable of generating a signal directly or indirectly.

The electron-donating group is linked, directly or indirectly, on one side to the electroactive polymer and on the other side to the antiligand. In this case, the electron-donating group is preferably linked covalently to the electroactive polymer via a first linking group, or else the electron-donating group is linked, directly or indirectly, to the antiligand, said antiligand itself being linked, directly or indirectly, to the electroactive polymer.

The antiligand is linked covalently to the electron-donating group via a second linking group and/or the antiligand is linked covalently to the electroactive polymer via a third linking group.

The first and/or second and/or third linking groups link, respectively, the electron-donating group to the electroactive polymer, the electron-donating group to the antiligand, and the antiligand to the electroactive polymer, via a coupling arm.

An advantageous probe of the invention comprises a polypyrrole formed from at least two monomers each consisting of a pyrrole ring, ferrocene and a particular antiligand consisting of a probe polynucleotide capable of hybridizing a target polynucleotide under appropriate hybridization conditions.

The probe polynucleotide (PN_{probe}) may be attached between the ferrocene (Fe) and the pyrrole ring (P) of said monomer at least, or else may be attached to the ferrocene, the latter being attached to the pyrrole ring.

When the probe polynucleotide is attached between the ferrocene and the pyrrole ring, in a structure that will be denoted by P-PN_{probe}-Fe, the probe of the invention has the following features taken individually or in combination:

the probe polynucleotide is attached to the carbon in the 1 position of one of the cyclopentadiene rings of the ferrocene;

the probe polynucleotide is attached, directly or indirectly, to the pyrrole ring via a third linking group; this is advantageously the —CH₂—CO-group.

When the probe polynucleotide is attached to the ferrocene which is itself attached to the pyrrole ring, in a structure that will be denoted by P-Fe-PN_{probe}, the probe of the invention has the following features taken individually or in combination:

the probe polynucleotide is attached to the carbon in the 1 position of one of the cyclopentadiene rings of the ferrocene, and the ferrocene is attached to the pyrrole ring via the carbon in the 1′ position of the other cyclopentadiene ring;

the ferrocene is attached, directly or indirectly, to the pyrrole ring of said monomer at least, via a first linking group; advantageously, the latter is the —CONH—CH₂— group.

Whatever the structure of the probe, the ferrocene is attached, directly or indirectly, to the probe polynucleotide via a second linking group which preferably consists of the —CO— group.

Moreover, the probe polynucleotide is attached to the pyrrole ring of the monomer at least and/or to the ferrocene via, respectively, the third and/or the second linking groups and via at least one of the amino functional groups of the probe polynucleotide.

According to an advantageous structure of a probe of the invention, the aforementioned first and/or second and/or third linking groups link, indirectly, the ferrocene to the pyrrole ring, the probe polynucleotide to the pyrrole ring and the probe polynucleotide to the pyrrole ring, respectively, via a coupling arm.

This coupling arm is preferably a saturated hydrocarbon chain having at least two carbon atoms, preferably at least 2 or 3 carbon atoms.

The modified polypyrrole according to the invention may be a homopolymer or a copolymer. The pyrrole ring is substituted on the carbon in the 3 position, and therefore any copolymer of the invention will have at least two monomers, at least one of which is not substituted in said position, or else it will have at least two monomers differently substituted in said position, the PN_{probe} substitutents being different and/or the combination of the PN_{probe} and Fe substitutents being different on the pyrrole ring of the monomers and/or the first and/or second and/or third linking groups and/or the coupling arms being different. In addition to the modified monomers according to the invention, a copolymer may include at least one monomer whose pyrrole ring is substituted with a —CH₂—COOH or —CH₂—CH₂OH group.

When all the monomers of the polymer are modified in an identical manner, a homopolymer polypyrrole of the invention is obtained.

The invention also relates to a method for preparing a probe of the invention.

If the modified monomers of the polypyrrole all have the P-PN_{probe}-Fe structure, the method then comprises the following steps:

(a) a homopolymer or copolymer polypyrrole formed by at least two monomers each consisting of a pyrrole ring, at least one of which is substituted on the carbon in the 3 position with a probe polynucleotide, is obtained;

(b) ferrocene having, on the carbon in the 1 position of one of the cyclopentadiene rings, at least one activated or activatable group is obtained; and

(c) the homopolymer or copolymer polypyrrole, substituted with a probe polynucleotide, is brought into contact with the ferrocene having said activated or activatable group.

If the modified monomers of the polypyrrole all have the P-Fe PN_{probe} structure, the process then comprises the following steps:

(a) ferrocene having at least two activated or activatable groups, at least one being attached to the carbon in the 1 position of one of the cyclopentadiene rings and the other being attached to the carbon in the 1′ position of the other cyclopentadiene ring of the ferrocene, is obtained;

(b) a monomer consisting of a pyrrole ring substituted on the carbon in the 3 position with an activated or activatable group is obtained;

(c) an excess of the ferrocene having activated or activatable groups is reacted with the substituted monomer;

(d) an electropolymerization of the compound obtained at (c) is carried out; and

(e) the polymer obtained in step (d) is brought into contact with a probe polynucleotide.

The probe preparation methods of the invention are furthermore advantageously characterized as follows:

the activated or activatable group or groups of the ferrocene, which may be identical or different, are preferably an activated or activatable ester group and preferably the —CO-[N-hydroxyphthalimide] group; advantageously, they are attached to the ferrocene via a coupling arm;

the activated group of the pyrrole ring is —CH₂—NH₂;

the activated or activatable group of the pyrrole ring is attached to the latter via a coupling arm;

a preferred coupling arm is a saturated hydrocarbon chain having at least two carbon atoms, preferably at least 2 or 3 carbon atoms.

The electropolymerization step is carried out using techniques well known to those skilled in the art. For example, it may be conducted by subjecting the monomers to electrical potential variations sufficient for the polymerization to take place successively by an oxidation and a reduction; or else by polymerization under an imposed current (chronopotentiometry) or under an imposed potential (chronoammetry).

Another subject of the invention is a ligand/ferrocene compound as intermediate product, consisting of a ligand linked, directly or indirectly, to an electron-donating group such as ferrocene, quinone or derivatives of these. In particular, this compound is a polynucleotide/ferrocene compound as an intermediate in the preparation of a probe of the invention in the method for preparing a P-PN_{probe}-Fe probe of the invention. It consists of a polynucleotide attached to the carbon in the 1 position of one of the cyclopentadiene rings of the ferrocene, via a second linking group which is preferably the —CO— group.

This second linking group may link the ferrocene and the probe polynucleotide indirectly, via a coupling arm which advantageously consists of a saturated hydrocarbon chain having at least two carbon atoms, preferably at least 2 or 3 carbon atoms.

A probe of the invention has diagnostic applications. Thus, the invention also relates to a method of detecting a target polynucleotide in a biological specimen, in which said probe is contacted, under appropriate hybridization conditions, and a difference in potential or a variation in current between the probe before contacting and the probe after contacting is demonstrated or quantified.

The invention also relates to the use of a probe for detecting a target polynucleotide in a biological specimen, in which use said probe is contacted, under appropriate hybridization conditions, and a difference in potential or a variation in current between the probe before contacting and the probe after contacting is demonstrated or quantified.

The subject of the present invention is also an electrode, all or part of the surface of which is coated with a probe defined above. Such an electrode may be obtained by any conventional technique well known to those skilled in the art. As an example, this preparation may be carried out by depositing a polypyrrole of the invention on the surface of an electrode made of platinum, gold, gold-coated chromium or titanium, glassy carbon or a conducting oxide such as tin oxide or a mixed indium-tin oxide.

The invention also relates to the use of an electron-donating group to increase the electroactivity of an electroactive polymer to which an antiligand capable of interacting with a ligand is attached, said electron-donating group being on the same monomer as the antiligand.

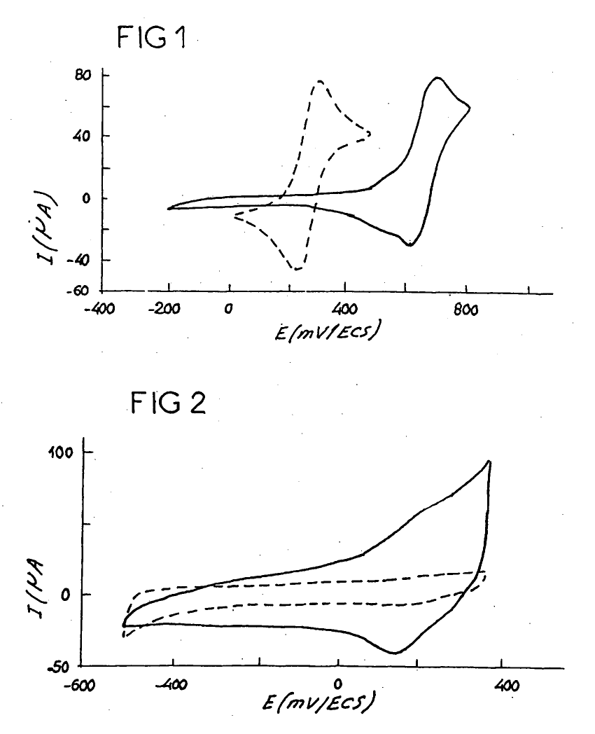

The various subjects of the invention are illustrated in the following examples which refer to the appended FIGS. 1 to 15 and from which their preferred and advantageous features will become apparent.

### EXAMPLE 1

### Preparation of a Copolymer Polypyrrole Consisting of Monomers Having a P-PN_{probe}-Fe Structure and of Monomers Substituted With the —CH₂—COOH group.

The production of this structure required the synthesis of a ferrocene substituted with an N-hydroxyphthalimide (NHP) group and the attachment of the probe polynucleotide to a polypyrrole, and then the attachment of the activated ferrocene obtained to the probe polynucleotide attached to the polypyrrole.

1A—Formation of the Activated Ferrocene: 1-ferrocene-propyl-NHP

The synthesis scheme was the following:

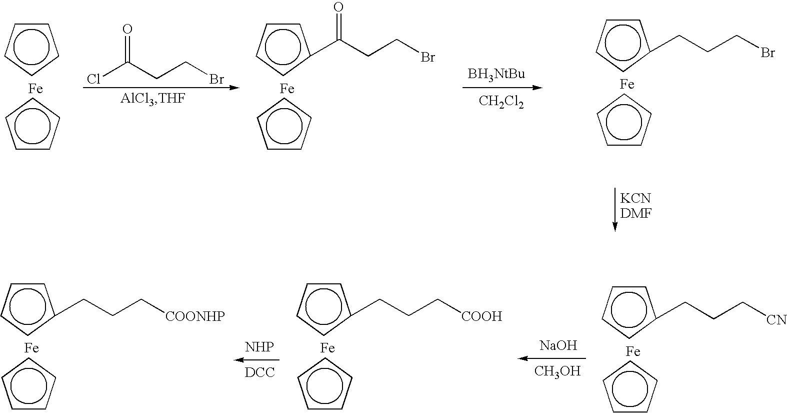

By analyzing the electrochemical response of the 1-ferrocene-propyl-NHP thus obtained, a reversible electrochemical system was demonstrated at a potential of 0.3 V/SCE. This reponse compared with that of a ferrocene substituted directly with an NHP group showed an increase in potential of around 200 mV.

1B—Attachment of the Probe Polynucleotide to the Copolymer Polypyrrole [PyCOOH,PyCONH—NHP]

This step was carried out under the same conditons as those described in document WO-A-95/29199, using a probe polynucleotide fuctionalized on its 3′ and 5′ ends by amino groups, one of the groups serving for attachment to the activated polypyrrole polymer and the other for attachement to the activated ferrocene.

1C—Attachment of the Ferrocene to the Copolymer Polypyrrole poly[PyCOOH,Py-PN_{probe}]

The grafting of the PN_{probe} was carried out by immersing a poly[PyCOOH,Py-PN_{probe}] electrode in a solution containing 1-ferrocene-propyl-NHP obtained at 1A, for 1 hour at room temperature. The electrode was then rinsed and electrochemically analyzed in a 0.5M NaCl solution.

The response shown in FIG. 1 (solid line: Fe—(CH₃)NHP; dotted line: Fe-NHP) is characterized by an electrochemical signal appearing, after grafting of the PN_{probe}, at a potential of 0.35 V/SCE in aqueous medium characteristic of the ferrocene substituted on the PN_{probe}. Measurement of the charge exchanged during the oxidation shows the presence of electroactive ferrocene sites. This measurement allows the total amount of ferrocene attached to the PN_{probe} to be determined.

### EXAMPLE 2

### Use of the Polypyrrole Obtained in Example 1 to Hybridize a Target Polynucleotide (P_{target})

The hybridization was carried out by placing the electrode covered with the polypyrrole obtained in example 1 for 1 hour in a PEG buffer in the presence of 25 nmol of PN_{target} at 37° C. The electrode was then rinsed and analyzed by cyclic voltammetry.

The electrochemical response shown in FIG. 2 (solid line: before hybridization; dotted line: after hybridization) shows a large decrease in the electroactivity of the signal from the ferrocene after hybridization. The current decreases by 70 μA. This large decrease demonstrates the great sensitivity of ferrocene to PN_{target} hybridization.

To optimize this polypyrrole, it is necessary to define a single site for grafting onto the PN_{probe} in order to control the position and the amount of ferrocene grafted, in order for the results to be perfectly reproducible. This optimization forms the subject of example 3.

### EXAMPLE 3

### Preparation of a Copolymer Polypyrrole Consisting of Monomers having the P-Fe-PN_{probe} structure

This preparation consisted in functionalizing the polypyrrole by a ferrocene carrying a leaving group. A PN_{probe} was attached, on one side, to the [PyNH-Fe-NHP] homopolymer and, on the other side, to the [PyNH-Fe-NHP-PyCOOH] copolymer.

The PN_{probe} has recognition properties with respect to a PN_{target} and it was checked whether this recognition was maintained after the PN_{probe} was attached to the ferrocene, and then the nature of the electrochemical response of the ferrocene to this recognition was sought.

Firstly, a pyrrole monomer substituted in the 3 position with a ferrocene carrying an activated ester group was synthesized. A polymerization of the monomer obtained was then carried out electrochemically and the PN_{probe} was grafted onto the resulting polymer.

3A—Synthesis of the 3-Pyrrole-Ferrocene-NHP Monomer

The production of a pyrrole carrying both a ferrocene and an NHP group required several synthesis steps. The first was the synthesis of a pyrrole substituted with an aminobutyl group, i.e. (4-aminobutyl)-3-pyrrole, the second step was the synthesis of 1,1′-dipropanoate-N-hydroxyphthalimide-ferrocene, and the third step was the synthesis of 3-pyrrole-ferrocene-NHP.

### Synthesis of (4-aminobutyl)-3-pyrrole

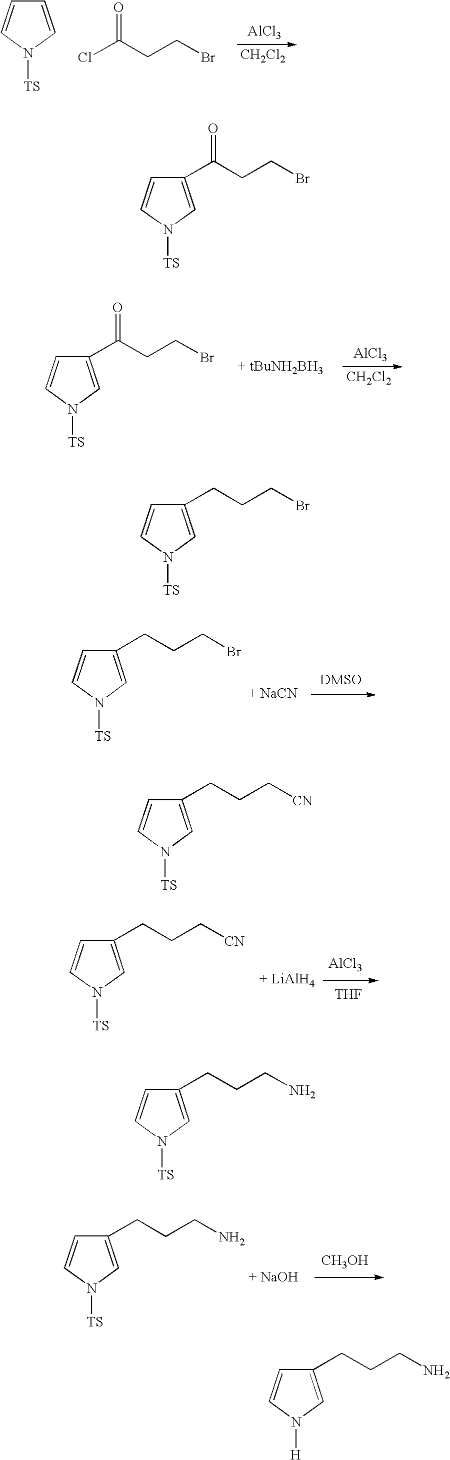

(4-Aminobutyl)-3-pyrrole was synthesized via the amino functional group allowing the pyrrole to be functionalized by activated ester groups by means of condensation reactions. To synthesize this monomer, an acylation reaction was carried out on tosyl-pyrrole in order to obtain (3-bromopropionyl)-3-tosylpyrrole with a yield of 63%. This reaction was followed by a reduction of the carbonyl functional group. (3-Bromopropyl)-1-tosylpyrrole was obtained with a yield of 78%. Using sodium cyanide, the bromine was substituted with a cyanide functional group with a yield of 50%. The conversion of the cyanide to an amine was performed by a reduction reaction using lithium aluminum hydride (77% yield). The hydrolysis of the tosyl group by an NaOH base resulted in (4-aminobutyl)-3-pyrrole with a yield of 62%.

Synthesis of 1,1′-ferrocene-propyl-NHP.

The synthesis scheme was the following:

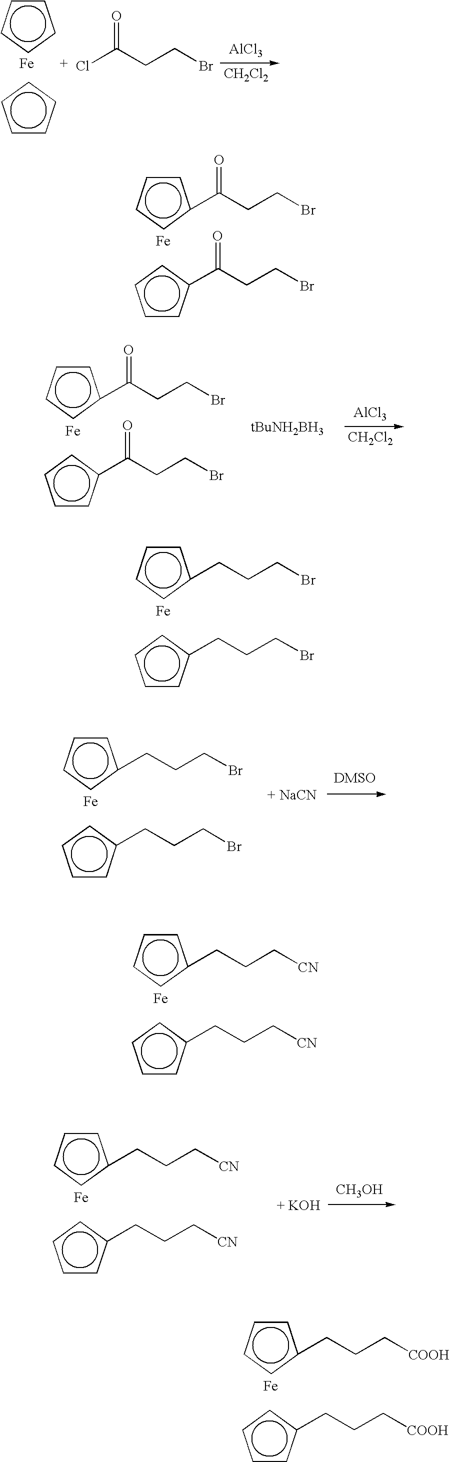

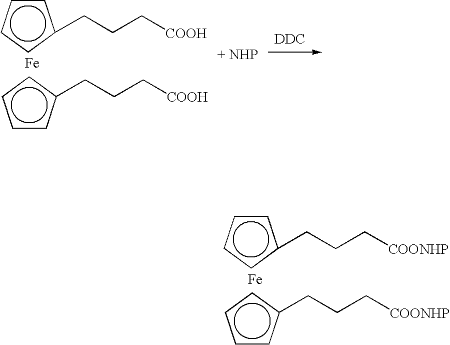

This ferrocene was obtained by the conversion of 1,1′-(cyanopropyl)ferrocene into 1,1′-(propyl)ferrocene acid in the presence of sodium hydroxide and methanol with a yield of 95%. The carboxylic groups of the ferrocene were substituted with a leaving group, N-hydroxyphthalimide (NHP). The NHP ferrocene was obtained from the ferrocene acid by esterification and in the presence of DCC. The DCC acted as a dehydrating agent and was converted into DCU, thus picking up the water formed during the reaction.

Synthesis of 3-pyrrole-ferrocene-NHP

The synthesis scheme was the following:

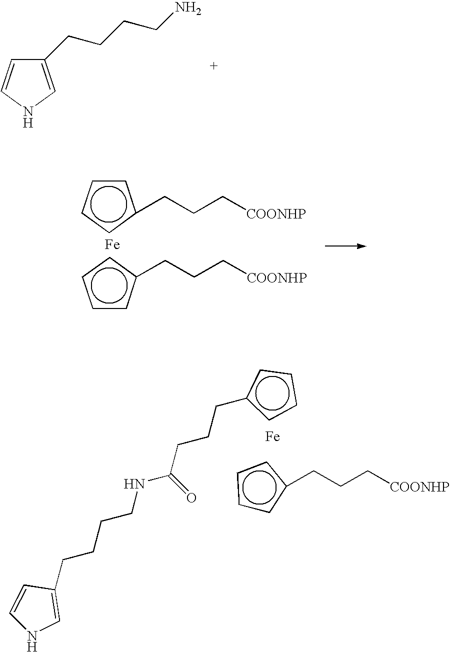

The 3-pyrrole-ferrocene-NHP was obtained by a coupling reaction between 3-pyrrole-butylamine and 1,1′-ferrocene-propyl-NHP in acetonitrile, under conditions of a large excess of ferrocene in order to avoid substituting the pyrrole on the two NHP groups of the ferrocene.

Electrochemical Characterization of the Monomer

The monomer was electrochemically analyzed in solution in acetonitrile in the presence of a 0.1M LiClO₄ support electrolyte.

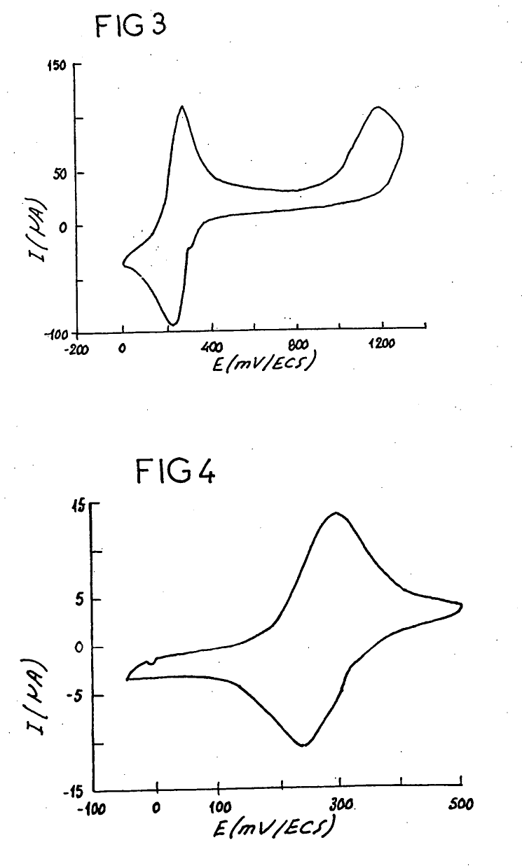

FIG. 3 shows the voltammogram obtained. Two oxidation-reduction systems may be seen. The first system has reversible characteristics, the oxidation potential of which is at 0.26 V. This system corresponds to the oxidation-reduction of ferrocene substituted on the pyrrole. The second electrochemical system is irreversible—its oxidation potential is at 1.17 V/SCE; it corresponds to the oxidation of the pyrrole.

This oxidation potential of the pyrrole substituted with the ferrocene group is high compared with the oxidation potential of unsubstituted pyrrole or pyrrole substituted with the carboxylic group, which is generally around 0.8 to 0.9 V/SCE.

3B—Preparation of the poly[Py-Fe-NHP] Homopolymer.

The polymerization scheme was the following:

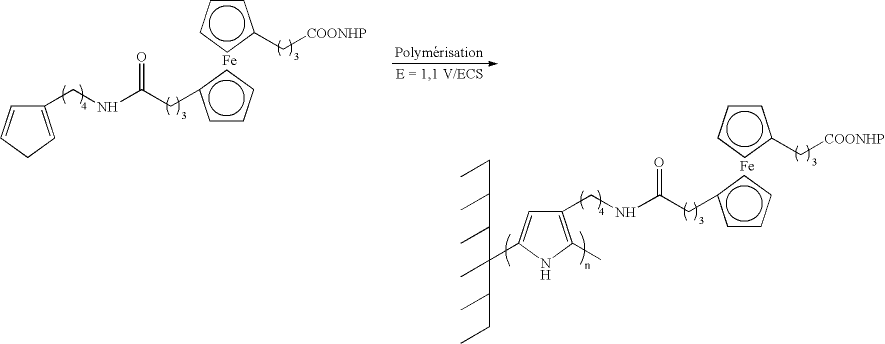

[Key to image on French page 18: Polymérization →polymerization 1,1 V/ECS →1.1 V/SCE]

The films of poly[Py-Fe-NHP] were obtained by immersing the platinum electrodes in a solution of the monomer poly[Py-Fe-NHP] in an acetonitrile medium containing 0.2M tetrabutylammonium tetrafluoroborate (N⁺BU₂BF₄). The electropolymerization was carried out at a controlled potential of 1.1 V/SCE, based on the oxidation potential of the monomer. The charge deposited was 70 mC.

After polymerization, a precipitate was formed in the solution, this probably being due to the formation of short-chain oligomers.

Electrochemical Characterization of the Poly[Py-Fe-NHP] Homopolymer

The films of poly[Py-Fe-NHP,PyCOOH] homopolymer obtained were electrochemically analyzed in an acetonitrile medium in the presence of 0.1M LiClO₄. FIG. 4 shows the voltammogram obtained. This voltammogram shows the high electroactivity of the polymer film. The oxidation potential is 288 mV/SCE and the reduction potential is 236 mV/SCE. The difference between these two values and the ratio of the oxidation peak current (13.5 μA) to the reduction peak current (11 μA) show that this electrochemical system exhibits good reversibility. The charge exchanged during the oxidation or the reduction is 0.1 mC. This value is very low compared with the charge deposited during the polymerization. From the oxidation charge, it is possible to determine the amount of electroactive ferrocene deposited on the electrode. Knowing that ferrocene exchanges one electron during its oxidation process, Faraday's law may be established:

Qₒₓ=N(Fe)*F.

The number of moles of ferrocene or of electroactive pyrrole units on the electrode was 10⁻⁹ mol.

Calculating the number of moles of ferrocene from the polymerization charge gives very high values of around 10⁻⁷ mol.

However, these results show that the polymerization yield is very low. Two factors are the cause of this low yield: on the one hand, some of the polymerization charge serves for oxidizing the ferrocene in solution and, on the other hand, the steric hindrance of the ferrocene-NHP group substituted on the pyrrole prevents coupling between these two pyrrole units and results in short-chain oligomers which precipitate in solution.

3C—Attachment of the PN_{probe} to the Homopolymer

The attachment scheme was the following:

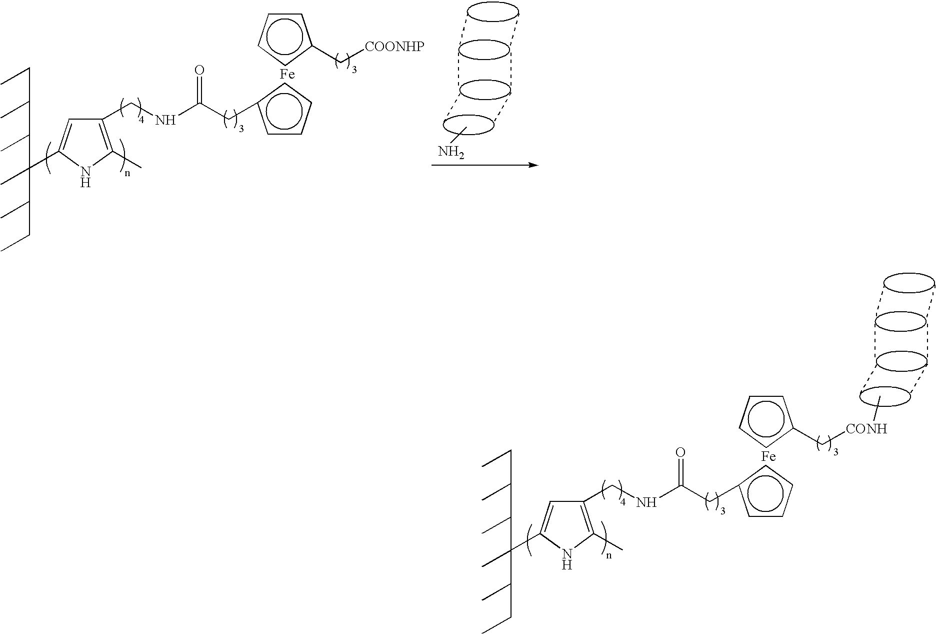

The PN_{probe} used was a polynucleotide having 25 pairs of bases with the following sequence identified by SEQ ID No. 1:

^{5′}TCA-ATC-TCG-GGA-ATC-TCA-ATG-TTA-G^{3′}.

The PN_{probe} was attached to the poly[Py-Fe-NHP] precursor homopolymer. The chemical conditions for this substitution were similar to those for attachment to the poly[PyCCNH-NHP] polymer.

The coupling between the pyrrole-Fe-NHP and the PN_{probe} carrying the amino group in its 5′ position was achieved by immersing the electrode in an acetonitrile solution in the presence of 10% of a 25 μmol/l acetate buffer. The reaction was carried out at room temperature for 2 hours.

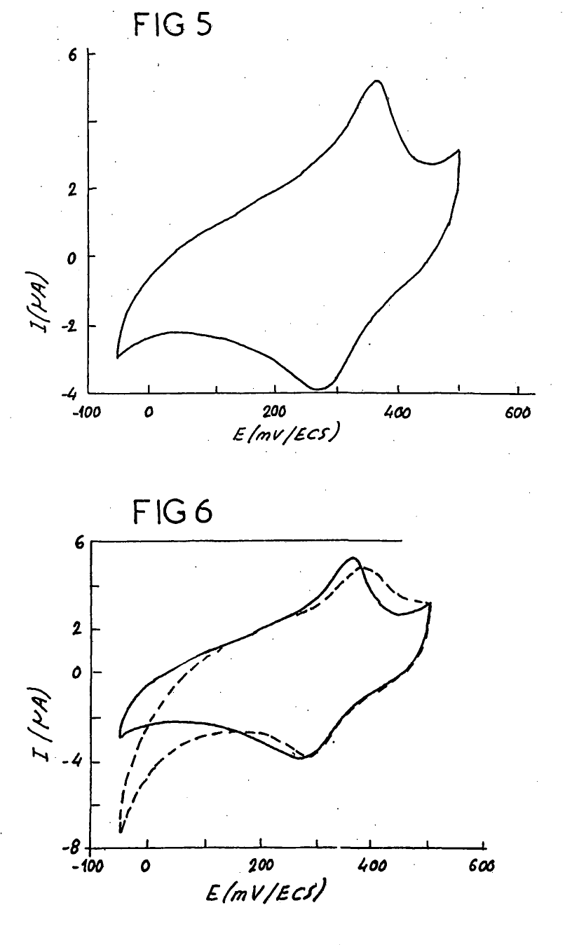

The films obtained were analyzed by cyclic voltammetry in water in the presence of 0.5M NaCl. FIG. 5 shows an oxidation-reduction system in which the oxidation and reduction peaks are located at 273 mV and 258 mV, respectively.

The electrochemical system of the ferrocene in aqueous medium is not as reversible compared with the analysis of the film in acetonitrile. This loss of electroactivity is probably due to problems of salvation either of the doping ion or of the polymer matrix in water.

### EXAMPLE 4

### Hybridization of the PN_{target} on the Poly[Py-Fe-PN_{probe}] Homopolymer Obtained in Example 3

The hybridization scheme was the following:

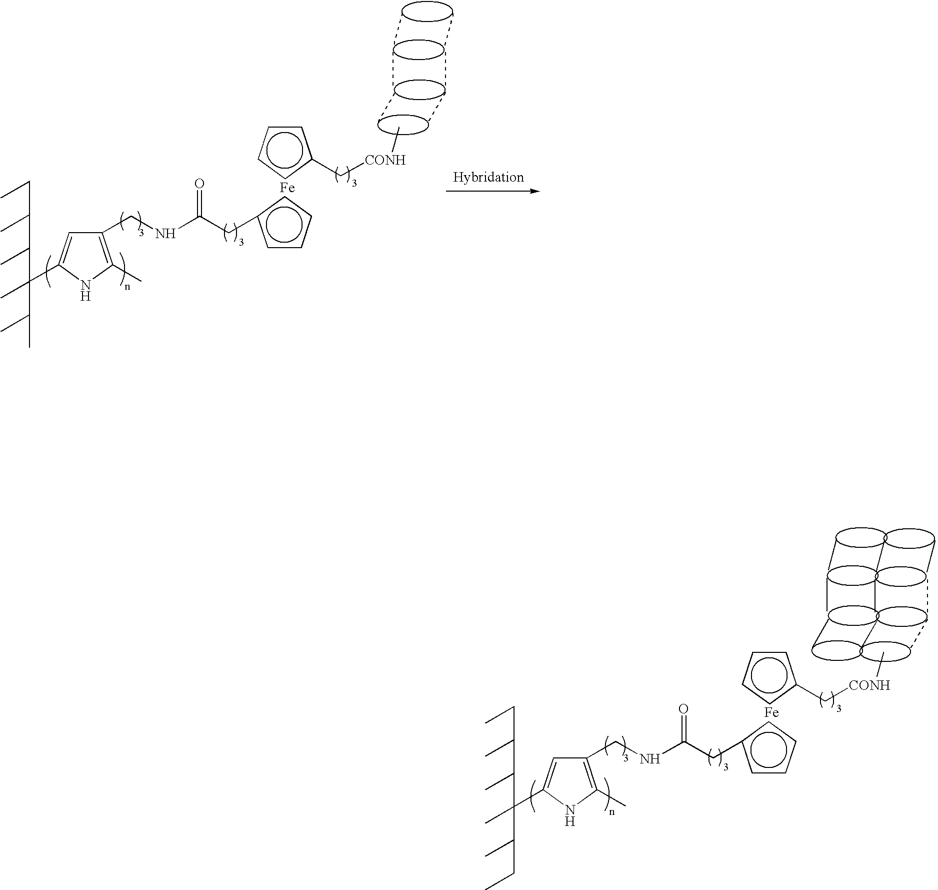

[Key to image on French page 20: Hybridation →Hybridization]

Films of poly[Py-Fe-PN_{probe}] copolymer were incubated in the presence of 25 nmol (25 μmol/l) of PN_{target} in a biological buffer, for 3 hours, at a temperature of 37° C. in a volume of 1 ml.

The PN_{target} had 25 bases complementary with the bases of the PN_{probe} and had the following sequence, identified by SEQ ID No. 2:

^{5′}CTA-ACA-TTG-AGA-TTC-CCG-AGA-TTG-A^{3′}.

A blank test was carried out by incubating another electrode in the hybridization buffer, which contained salmon DNA in order to determine the effects of the nonspecific interactions.

Electrochemical Characterization

The electrochemical response of the polymers was analyzed by cyclic voltammetry in aqueous medium in the presence of 0.5M NaCl.

Blank-Test Electrode

The voltammogram given in FIG. 6 (solid line: before hybridization; dotted line: after hybridization) shows the electrochemical response of the control electrode incubated in the presence of PEG buffer.

It may be seen that the electrode, acting as control electrode, shows a variation in the oxidation potential and a decrease in the electroactivity. This indicates that the nonspecific interactions have a slight effect on the electrochemical response of ferrocene.

Polymer Tested After Hybridization

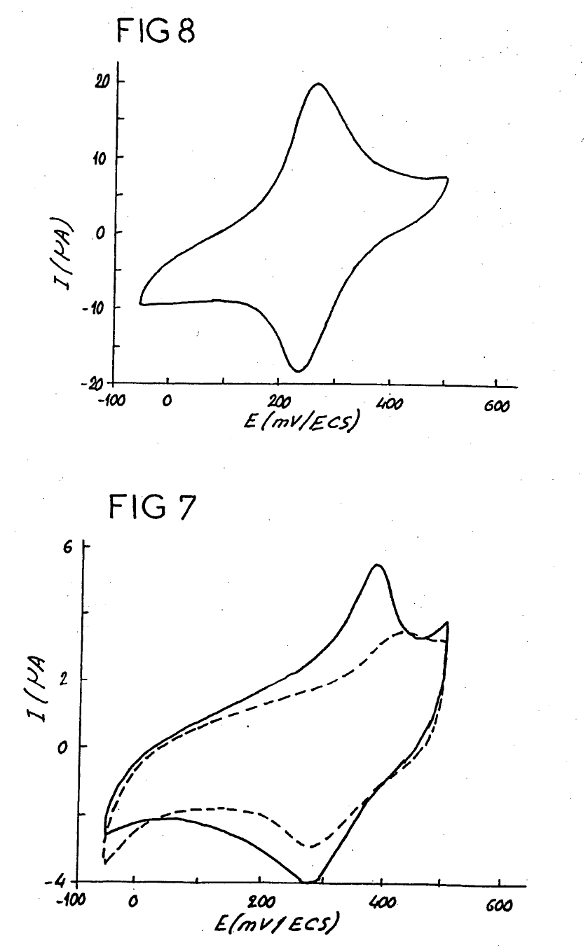

The electrochemical analysis of the electrode incubated in the presence of the PN_{target} in aqueous medium is shown in FIG. 7 (solid line: before hybridization; dotted line: after hybridization).

The electrode after incubation shows a shift in the oxidation potential toward high potentials and a decrease in the electroactivity. The charge exchanged in the course of the oxidation is of the reduction also decreases.

It may be seen that the variation in the electrochemical reponse of the ferrocene is greater compared with the blank electrode.

### EXAMPLE 5

### Preparation of a Copolymer Polypyrrole Consisting of Monomers Having the P-Fe-NHP Structure and of Monomers Substituted With the —CH₂—COOH Group

5A—Preparation of the poly[Py-Fe-NHP,PyCOOH] polymer

The synthesis scheme was the following:

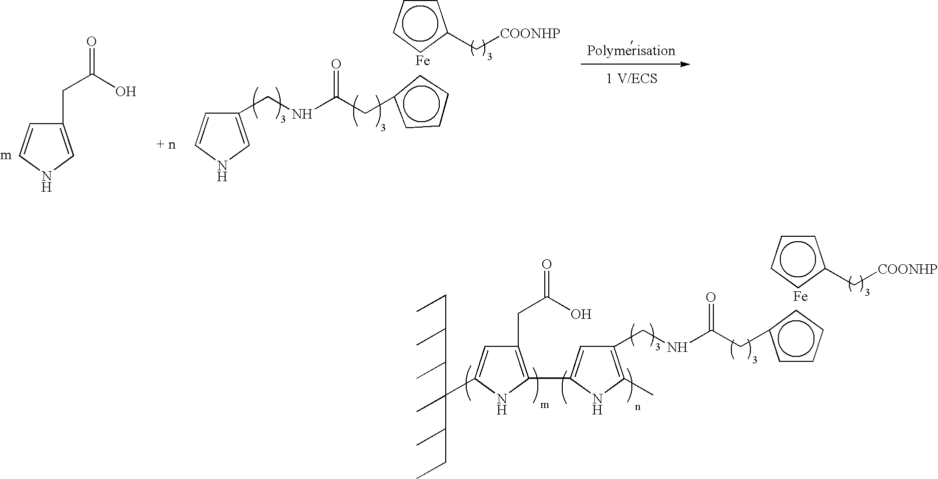

[Key to image on French page 22: Polymérisation →Polymerization 1 V/ECS →1 V/SCE]

The copolymerization between 3-pyrrole acetic acid (PyCOOH) and 3-pyrrole-Fe-NHP (Py-Fe-NHP) made it possible to dilute this bulky monomer in the polymer matrix so as to minimize the sterically induced perturbations. Furthermore, copolymerization with 3-pyrrole acetic acid acid made it possible to increase the porosity of the films.

The choice of respective concentrations of the PyCOOH and Py-Fe-NHP monomers was determined by referring to the polymerization potentials for these two monomers. PyCOOH oxidizes at 0.9 V/SCE while Py-Fe-NHP oxidizes at 1.1 V/SCE. It was necessary to take a small concentration of PyCOOH compared with Py-Fe-NHP. The concentrations of these two monomers used for the electropolymerization of the copolymer were therefore 0.02M in the case of PyCOOH and 0.08M in the case of Py-Fe-NHP.

The copolymer was deposited at an imposed potential of 1.1 V/SCE on a platinum electrode having an area of 700 cm² in a propylene carbonate medium in the presence of 0.2M of LiCl₂. The charge deposited during the electropolymerization was 35 mC. It was difficult to determine the thickness of the film from the polymerization charge since some of the charge measured during the electropolymerization was used to oxidize the ferrocene in solution.

Electrochemical Characterization of the Poly[Py-Fe-NHP,PyCOOH] Polymer Obtained

This characterization was carried out in an acetonitrile medium in the presence of 0.1 M LiClO₄. The voltammogram in FIG. 8 shows a reversible electrochemical system with an oxidation peak at a potential of 254 mV and a symmetrical reduction peak at a potential of 234 mV. The mid-height width is 150 mV. This electrochemical response presents the signal from the ferrocene and shows that this electrochemical probe has a high electroactivity. Electron transfer along the conjugated chain of the polymer is very extensive, which shows that the polypyrrole is conductive. The charge echanged during oxidation of the ferrocene was around 0.15 mC. This ferrocene oxidation charge allows the number of moles of pyrrole carrying the ferrocene group in the polymer to be calculated, assuming that all the ferrocene sites are electroactive.

From Faraday's law:

Qₒₓ=N*F

where N is the number of moles of ferrocene and F is Faraday's constant.

The number of moles of electroactive ferrocene is around 10⁻⁹ mol.

5B—Attachment of the Probe Polynucleotide to the poly[Py-Fe-NHP,PyCOOH] Copolymer Polypyrrole

The attachment scheme was the following:

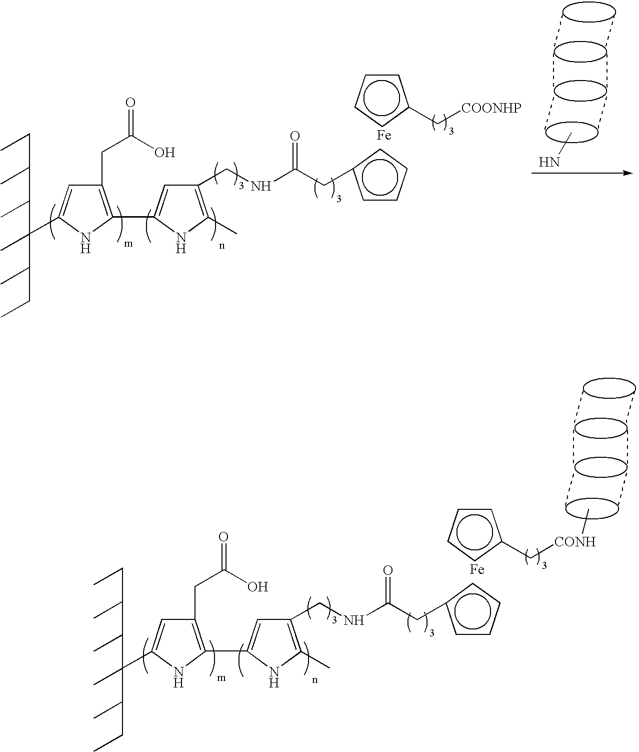

The PN_{probe} used was the polynucleotide SEQ ID No. 1.

The PN_{probe} (25 μmol/l) was attached to the poly[Py-Fe-NHP,PyCOOH] precursor copolymer by immersing the electrode in an acetonitrile solution in the presence of 10% of acetate buffer.

Electrochemical Characterization of the Poly[Py-Fe-PN_{Probe}, PyCOOH] Polymer Obtained

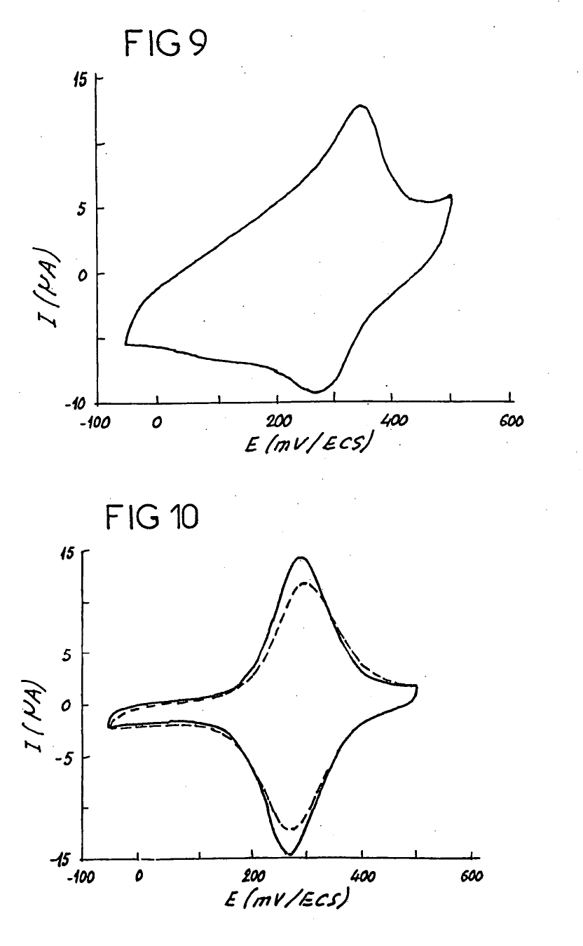

FIG. 9 shows the electrochemical response of the electrode after grafting the PN_{probe} in an aqueous medium in the presence of 0.5M NaCl. The voltammogram shows an oxidation-reduction system characterized by an intense narrow oxidation peak and a broad reduction peak.

The potentials of the oxidation and reduction peaks are 341 mV and 275 mV respectively, the mid-height width is 100 mV in the case of the oxidation peak and 200 mV in the case of the reduction peak. The charge exchanged during the oxidation or the reduction was around 0.1 mC.

### EXAMPLE 6

### Hybridization of the PN_{target} on the Poly[Py-Fe-PN_{probe},PyCOOH] Copolymer Obtained in Example 5

The hybridization scheme was the following:

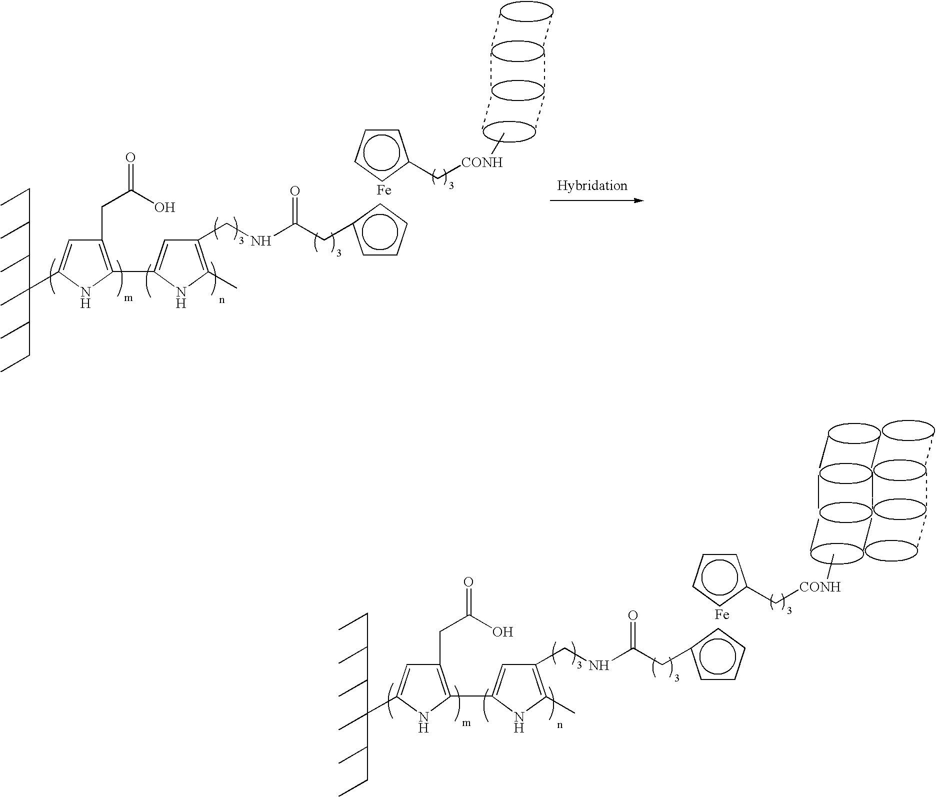

[Key to image on French page 24: Hybridation →Hybridization]

Films of poly[Py-Fe-PN_{probe},PyCOOH] copolymer were incubated in the presence of PN_{target} consisting of 25 bases under the same conditions as previously.

A control electrode was incubated in the same biological buffer without the PN_{target}.

Electrochemical Characterization

The electrochemical response of the electrodes incubeated in the presence of the target and of the non-target was analyzed in acetonitrile medium and in aqueous medium.

Blank-Test Electrode

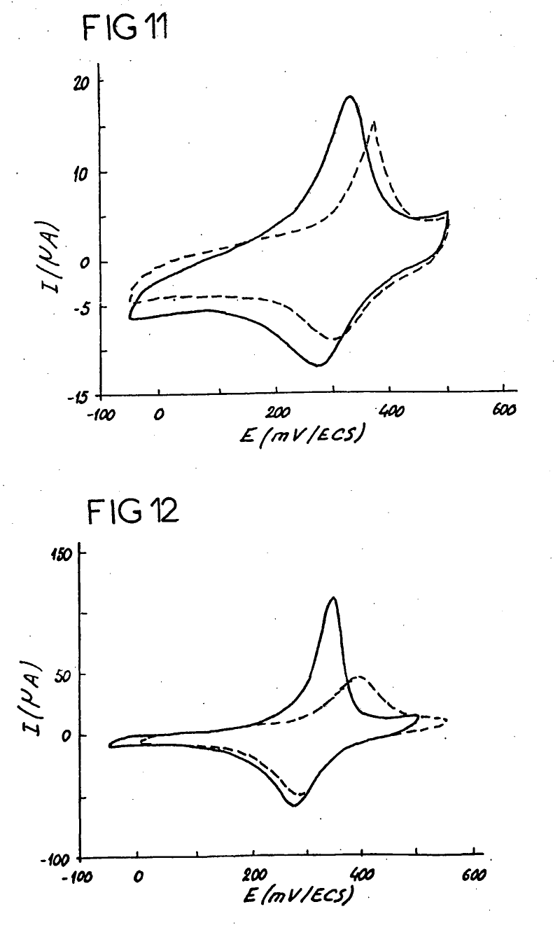

FIGS. 10 and 11 show the voltammograms before (solid line) and after (dotted line) hybridization in the presence of the non-target (salmon DNA) in acetonitrile medium and in aqueous medium.

It may be seen that, in acetonitrile medium, the electrochemical response of the ferrocene shows that the oxidation potential is stable, with a very slight variation in the peak currents.

Analysis of the electrochemical response of the control electrode in aqueous medium in the presence of 0.5M NaCl shows a variation in the electrochemical response of the ferrocene—an increase in the oxidation potential and a decrease in the peak current.

Copolymer Tested After Hybridization

The electrochemical analysis of the electrode incubated in the presence of the target in aqueous medium is shown in FIG. 12 (solid line: before hybridization; dotted line: after hybridization). The voltammogram shows a large variation in the electrochemical signal after hybridization. The oxidation potential is shifted toward the higher potentials; it goes from 340 mV to 387 mV after hybridization. The oxidation current decreases by 60%. The charge exchanged during the oxidation decreases by 25%.

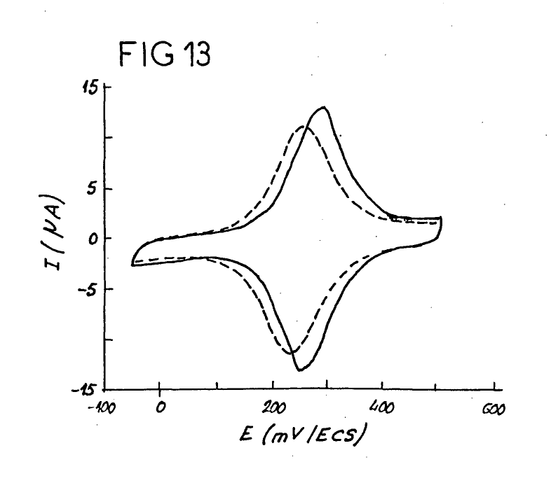

Analysis of the electrochemical response in acetonitrile of the electrode incubated in the presence of 0.1 nmol of PN_{target} is shown in FIG. 13 (solid line: before hybridization; dotted line: after hybridization). The voltammogram after incubation shows that the reversible system of the ferrocene in organic medium is maintained. The oxidation and reduction potential of the ferrocene is shifted toward the low potentials.

This variation in the electrochemical response in aqueous medium and in organic medium demonstrates that there is selective and specific hybridization between the PN_{probe} grafted onto the ferrocene and the PN_{target}.

Although these electrodes incubated in the presence of the target showed significant variations in the electrochemical response, it is necessary to determine the origin of the variation in the reponse of the ferrocene after incubation in a biological buffer without the target.

### EXAMPLE 6

### Influence of the Polymerization Charge on Electroactivity

The influence of the charge deposited during the polymerization on the variation in the electrochemical signal from the poly[Py-Fe-PN_{probe},PyCOOH] of example 5 was studied for three charge values, 5, 10 and 15 mC.

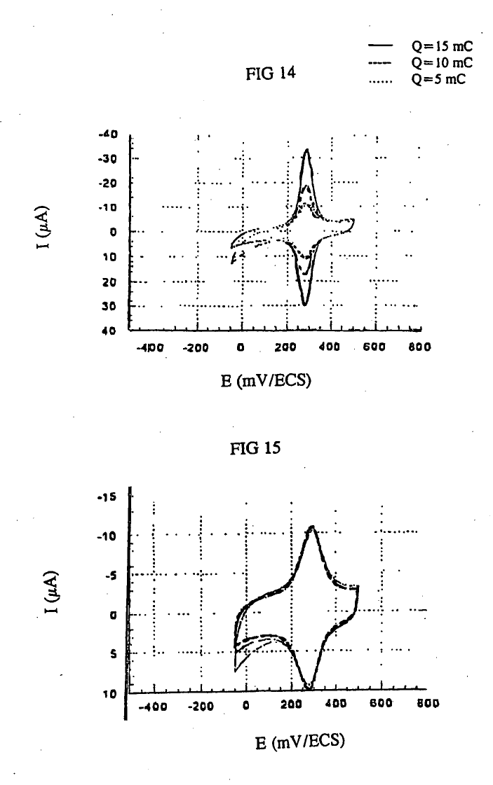

FIG. 14 demonstrates that the oxidation current decreases with the amount of charge deposited.

### EXAMPLE 7

### Electrochemical Reproducibility of the Electrodes

The electrochemical reproducibility of the poly[Py-Fe-PN_{probe},PyCOOH] of example 5 was tested on six thin film electrodes.

The electrochemical characterization of these electrodes, performed in acetonitrile medium in the presence of 0.1M LiClO₄, is illustrated in FIG. 15, which shows that for the six electrodes substantially the same voltammograms are obtained after the PN_{probe} has been grafted.

### EXAMPLE 8

### Influence of the PN_{target} Hybridization Conditions on the PN_{probe}

8A—Effect of the Hybridization Buffer

The same poly[Py-Fe-PN_{probe},PyCOOH] (Q=5 mC) electrode as in example 5 was incubated in two different buffers:

TE-NaCl buffer at 37° C. for 2 hours and 3 hours;

SSPEG buffer at 37° C. for 2 hours and 3 hours.

The electrochemical characterization (the voltammograms are non shown) carried out in acetonitrile medium in the presence of 0.1M LiClO₄, respectively, before and after the incubation, for each of the above buffers demonstrated the benefit of the SSPEG buffer in which the electrode is more stable.

8B—Effect of the PN_{target} Concentration

Several poly[Py-Fe-PN_{probe},PyCOOH] films of example 5 were incubated in an SSPEG buffer in the presence of various PN_{target} concentrations (0.01, 0.05 and 0.5 nmol/l).

The electrochemical characterization (the voltammograms are not shown) performed in acetonitrile medium in the presence of 0.1M LiClO₄, before and after hybridization respectively, in the presence of the various concentrations above showed that the variation in the electrochemical response before and after hybridization increased with the PN_{target} concentration. This is because it was observed that, at a potential of 280 mV/SCE, which corresponds to the potential of the initial peak, the decrease in current varies with the PN_{target} concentration.

1. An electroactive complex consisting of an electroactive, homopolymer or copolymer, polymer of at least two monomers, an antiligand and a ligand that has specifically interacted with said antiligand, characterized in that said antiligand is linked, directly or indirectly, to the electroactive polymer and in that it furthemore includes at least one electron-donating group linked, directly or indirectly, to the antiligand.

2. The complex as claimed in claim 1, characterized in that the electroactive polymer is chosen from polypyrrole, polyacetylene, polyazine, poly(p-phenylene), poly(p-phenylene vinylene), polypyrene, polythiophene, polyfuran, polyselenophene, polypyridazine, polycarbazole, polyaniline and double-stranded polynucleotides.

3. The complex as claimed in claim 1, characterized in that the electron-donating group is chosen from ferrocene, quinone and derivatives of these.

4. The complex as claimed in claim 1, characterized in that the ligand and the antiligand are biological molecules especially chosen from polynucleotides and polypeptides.

5. The complex as claimed in claim 4, characterized in that the ligand and/or the antiligand are labeled by a tracer capable of generating a signal directly or indirectly.

6. The complex as claimed in claim 1, characterized in that the electron-donating group is linked, directly or indirectly, to the ligand that has interacted with the antiligand, said antiligand being linked, directly or indirectly, to the electroactive polymer.

7. The complex as claimed in claim 6, characterized in that the antiligand or the ligand are linked covalently to the electron-donating group via a second linking group.

8. The complex as claimed in claim 1, characterized in that the antiligand or the ligand are linked covalently to the electroactive polymer via a third linking group.

9. The complex as claimed in either of claims 7 and 8, characterized in that the first and/or second and/or third linking groups link, respectively, the electron-donating group to the antiligand and the antiligand to the electroactive polymer, via a coupling arm.

10. The complex as claimed in claim 6, characterized in that the electron-donating group is linked to the ligand via an inert or biological support.

11. The complex as claimed in claim 10, characterized in that the inert support is a polystyrene bead, a magnetic bead or a glass bead.

12. The complex as claimed in claim 10, characterized in that the biological support is a cell in which the electron-donating group has been internalized.

13. The complex as claimed in claim 1, characterized in that the electroactive polymer is a polypyrrole formed from at least two monomers each consisting of a pyrrole ring and in that the electron-donating group is ferrocene.

14. The complex as claimed in claim 13, characterized in that the antiligand is a probe polynucleotide and the ligand is a target polynucleotide, at least partly hybridized with said antiligand.

15. The complex as claimed in claim 14, characterized in that the probe polynucleotide is linked to the ferrocene and to the pyrrole ring of said monomer at least.

16. The complex as claimed in claim 13, characterized in that the pyrrole ring is substituted on the carbon in the 3 position.

17. The complex as claimed in claim 15, characterized in that the probe polynucleotide is attached to the carbon in the 1 position of one of the cyclopentadiene rings of the ferrocene.

18. The complex as claimed in claim 13, characterized in that the polypyrrole is a copolymer and comprises a monomer whose pyrrole ring is substituted with a —CH₂—COOH or —CH₂—CH₂OH group.

19. The complex as claimed in claim 15, characterized in that the second linking group between the ferrocene and the probe polynucleotide or target polynucleotide is the —CO— group.

20. The complex as claimed in claim 15, characterized in that the third linking group between the probe polynucleotide or target polynucleotide and the pyrrole ring is the —CH₂—CO— group.

21. The complex as claimed in either of claims 19 and 20, characterized in that the first and/or second and/or third linking groups link, indirectly, the ferrocene to the probe polynucleotide or target polynucleotide, and the pyrrole ring to the probe polynucleotide or target polynucleotide, via a coupling arm.

22. The complex as claimed in claim 21, characterized in that the coupling arm is a saturated hydrocarbon chain having at least two carbon atoms, preferably at least 2 or 3 carbon atoms.

23. The complex as claimed in any one of claims 14 to 22, characterized in that the probe polynucleotide or the target polynucleotide is attached to the ferrocene and/or to the pyrrole ring of the monomer at least, via, respectively, the second and/or the third linking groups and via at least one of the amino functional groups of the probe polynucleotide or target polynucleotide.

24. An electroactive probe consisting of an electroactive, homopolymer or copolymer, polymer of at least two monomers, an antiligand and a ligand that has specifically interacted with said antiligand, characterized in that said antiligand is linked, directly or indirectly, to the electroactive polymer and in that it furthermore includes at least one electron-donating group linked, directly or indirectly, to the antiligand.

25. The probe as claimed in claim 24, characterized in that the electroactive polymer is chosen from polypyrrole, polyacetylene, polyazine, poly(p-phenylene), poly(p-phenylene vinylene), polypyrene, polythiophene, polyfuran, polyselenophene, polypyridazine, polycarbazole, polyaniline and double-stranded polynucleotides.

26. The probe as claimed in claim 24, characterized in that the electron-donating group is chosen from ferrocene, quinone and derivatives of these.

27. The probe as claimed in claim 24, characterized in that the ligand is a biological molecule especially chosen from polynucleotides and polypeptides.

28. The probe as claimed in claim 27, characterized in that the ligand is labeled by a tracer capable of generating a signal directly or indirectly.

29. The probe as claimed in claim 24, characterized in that the antiligand is linked covalently to the electron-donating group via a second linking group.

30. The probe as claimed in claim 24, characterized in that the antiligand is linked covalently to the electroactive polymer via a third linking group.

31. The probe as claimed in any one of claims 37 to 39, characterized in that the first and/or second and/or third linking groups link, respectively, the electron-donating group to the antiligand, and the antiligand to the electroactive polymer, via a coupling arm.

32. The probe as claimed in claims 25 and 26, characterized in that the electroactive polymer is polypyrrole formed from at least two monomers each consisting of a pyrrole ring and in that the electron-donating group is ferrocene.

33. The probe as claimed in claims 24 and 32, characterized in that the antiligand is a probe polynucleotide capable of hybridizing a target polynucleotide under appropriate hybridization conditions.

34. The probe as claimed in claim 33, characterized in that the probe polynucleotide is linked to the ferrocene and to the pyrrole ring of said monomer at least.

35. The probe as claimed in claim 32, characterized in that the pyrrole ring is substituted on the carbon in the 3 position.

36. The probe as claimed in claim 34, characterized in that the probe polynucleotide is attached to the carbon in the 1 position of one of the cyclopentadiene rings of the ferrocene.

37. The probe as claimed in claim 32, characterized in that the polypyrrole is a copolymer and comprises a monomer whose pyrrole ring is substituted with a —CH₂—COOH or —CH₂—CH₂OH group.

38. The probe as claimed in claim 34, characterized in that the second linking group between the ferrocene and the probe polynucleotide is the —CO— group.

39. The probe as claimed in claim 34, characterized in that the third linking group between the probe polynucleotide and the pyrrole ring is the —CH₂—CO— group.

40. The probe as claimed in either of claims 38 and 39, characterized in that the first and/or second and/or third linking groups link, indirectly, the ferrocene to the probe polynucleotide, and the pyrrole ring to the probe polynucleotide, respectively, via a coupling arm.

41. The probe as claimed in claim 40, characterized in that the coupling arm is a saturated hydrocarbon chain having at least two carbon atoms, preferably at least 2 or 3 carbon atoms.

42. The probe as claimed in any one of claims 32 to 41, characterized in that the probe polynucleotide is attached to the ferrocene and/or to the pyrrole ring of the monomer at least, via, respectively, the second and/or the third linking group and via at least one of the amino functional groups of the probe polynucleotide.

43. A method for preparing a probe as claimed in claim 31, characterized in that it comprises the following steps:
(a) a homopolymer or copolymer polypyrrole formed by at least two monomers each consisting of a pyrrole ring, at least one of which is substituted on the carbon in the 3 position with a probe polynucleotide, is obtained;
(b) ferrocene having, on the carbon in the 1 position of one of the cyclopentadiene rings, at least one activated or activatable group is obtained; and
(c) the homopolymer or copolymer polypyrrole, substituted with a probe polynucleotide, is brought into contact with the ferrocene having said activated or activatable group.

44. The method as claimed in claim 43, characterized in that the activated or activatable group or groups of the ferrocene, which may be identical or different, are an activated or activatable ester group and preferably a —CO—[N-hydroxyphthalimide] group.

45. The method as claimed in either of claims 43 and 44, characterized in that the activated or activatable group or groups of the ferrocene are attached to the latter via a coupling arm.

46. A method of detecting a ligand in a biological specimen, characterized in that a probe as claimed in any one of claims 24 to 42 is contacted under appropriate reaction conditions for the specific antiligand/ligand interaction and in that a difference in potential or a variation in current between the probe before contacting and the probe after contacting is demonstrated or quantified.

47. The method as claimed in claim 46, characterized in that the ligand is a polynucleotide.

48. An electrode, all or part of the surface of which is coated with a probe as claimed in any one of claims 24 to 42.

49. The use of an electron-donating group to increase the electroactivity of an electroactive polymer to which an antiligand capable of interacting with a ligand is attached, said electron-donating group being on the same monomer as the antiligand.

47. A method of detecting a ligand in a biological specimen, characterized in that an electroactive probe is contacted, said probe consisting of an electroactive, homopolymer or copolymer, polymer of at least two monomers and an antiligand capable of interacting specifically with a ligand, said probe furthermore including at least one electron-donating group linked, directly or indirectly, on one side to the electroactive polymer and on the other side to the antiligand, under reaction conditions appropriate for the specific antiligand/ligand interaction, and in that a difference in potential or a variation in current between the probe before contacting and the probe after contacting is demonstrated or quantified.

48. The method as claimed in claim 47, characterized in that the electroactive polymer is chosen from polypyrrole, polyacetylene, polyazine, poly(p-phenylene), poly(p-phenylene vinylene), polypyrene, polythiophene, polyfuran, polyselenophene, polypyridazine, polycarbazole, polyaniline and double-stranded polynucleotides.

49. The method as claimed in claim 47, characterized in that the electron-donating group is chosen from ferrocene, quinone and derivatives of these.

50. The method as claimed in claim 47, characterized in that the ligand is a biological molecule especially chosen from polynucleotides and polypeptides.

51. The method as claimed in claim 47, characterized in that the ligand is labeled by a tracer capable of generating a signal directly or indirectly.

52. The method as claimed in claim 47, characterized in that the electron-donating group is linked, directly or indirectly, on one side to the electroactive polymer and on the other side to the antiligand.

53. The method as claimed in claim 47, characterized in that the electron-donating group is linked covalently to the electroactive polymer via a first linking group.

54. The method as claimed in claim 47, characterized in that the antiligand is linked covalently to the electron-donating group via a second linking group.

55. The method as claimed in any one of claims 52 to 54, characterized in that the first and/or second and/or third linking groups link, respectively, the electron-donating group to the electroactive polymer and the electron-donating group to the antiligand, via a coupling arm.

56. The method as claimed in claims 49 and 50, characterized in that the electroactive polymer is the polypyrrole formed from at least two monomers each consisting of a pyrrole ring and in that the electron-donating group is ferrocene.

57. The method as claimed in claims 47 and 56, characterized in that the antiligand is a probe polynucleotide capable of hybridizing a target polynucleotide under appropriate hybridization conditions.

58. The method as claimed in claim 47, characterized in that the ferrocene is linked on one side to the probe polynucleotide and on the other side to the pyrrole ring of a monomer of the polypyrrole.

59. The method as claimed in claim 56, characterized in that the pyrrole ring is substituted on the carbon in the 3 position.

60. The method as claimed in claim 58, characterized in that the probe polynucleotide is linked to the carbon in the 1 position of one of the cyclopentadiene rings of the ferrocene.

61. The method as claimed in claim 58, characterized in that the probe polynucleotide is attached to the carbon in the 1 position of one of the cyclopentadiene rings of the ferrocene and the ferrocene is attached to the pyrrole ring via the carbon in the 1′ position of the other cyclopentadiene ring.

62. The method as claimed in claim 56, characterized in that the polypyrrole is a copolymer and comprises a monomer whose pyrrole ring is substituted with a —CH₂—COOH or —CH₂—CH₂OH group.

63. The method as claimed in claim 55, characterized in that the first linking group between the ferrocene and the pyrrole ring is the —CONH—CH₂— group.

64. The method as claimed in claim 55, characterized in that the second linking group between the ferrocene and the probe polynucleotide is the —CO— group.

65. The method as claimed in either of claims 63 and 64, characterized in that the first and/or second and/or third linking groups link, indirectly, the ferrocene to the pyrrole ring and the ferrocene to the probe polynucleotide, respectively, via a coupling arm.

66. The method as claimed in any one of claim 65, characterized in that the coupling arm is a saturated hydrocarbon chain having at least two carbon atoms, preferably at least 2 or 3 carbon atoms.

67. The probe as claimed in any one of claims 56 to 66, characterized in that the probe polynucleotide is attached to the ferrocene via, respectively, the second and/or the third linking group and via at least one of the amino functional groups of the probe polynucleotide.
